(19) United States
(12) Patent Application Publication
(10) Pub. No.: US 2005/0273147 A1
(43) Pub. Date: Dec. 8, 2005

(54) Expandable and contractible guidewire

(76) Inventor: Henry M. Israel, Bnei Brak (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/857,870

(22) Filed: Jun. 2, 2004

(51) Int. Cl. A61F 2/06

(52) U.S. Cl. 623/1.11

(57) ABSTRACT

A guidewire for a stent assembly, the guidewire including an outwardly expandable and inwardly contractible deformable portion.

### FIELD OF THE INVENTION

The present invention relates generally to stents, and particularly to a guidewire that facilitates implanting bifurcated stents in a body.

### BACKGROUND OF THE INVENTION

A stent is a well known device used to support an intraluminal wall, used in procedures, such as but not limited to, percutaneous transluminal coronary angioplasty (PTCA). Various types of stent architectures are known in the art, including braided stents (filaments or wires, wound or braided into a particular configuration), or mesh stents (metal mesh bent or formed into a particular shape), among others.

Typically, a stent may be restrained in a radially compressed configuration by a sheath or catheter, and delivered by an introducer to the site where it is required. The introducer may pass over a guidewire (like a monorail) that has been entered through the patient's skin, or through a blood vessel exposed by minor surgical means. When the introducer has been threaded into the body lumen to the stent deployment location, the introducer is manipulated to cause the stent to be released. The stent expands to a predetermined diameter at the deployment location, and the introducer is withdrawn. Stent expansion may be effected by spring elasticity, balloon expansion, or by the self-expansion of a thermally or stress-induced return of a shape memory alloy (such as a nickel-titanium alloy, e.g., NITINOL) to a pre-conditioned expanded configuration.

There are bifurcated lumens, such as but not limited to, the carotid artery, which may need support with a bifurcated stent. A bifurcated lumen (also called bifurcation) is an area of the vasculature where a first vessel is bifurcated into two or more branch vessels. Stenotic lesions may form in or around such bifurcations, that is, in or around one or more of the vessels.

However, delivering and deploying a stent to support a bifurcated lumen is a difficult challenge. Some of the problems include the difficulty of properly orienting the stent with respect to the bifurcation and the difficulty of providing a stent that supports the main trunk and branches of the bifurcation without blocking the passageways or causing turbulence or other flow disruptions.

PCT patent application PCT/IL03/00814 to Henry Israel, describes a bifurcated stent assembly with a stent sheath that includes two individually removable portions, one removable in a distal direction and the other in a proximal direction.

### SUMMARY OF THE INVENTION

The present invention seeks to provide a guidewire that facilitates implanting bifurcated stents in a body, and which may be particularly useful in deployment of the bifurcated stent assembly of PCT patent application PCT/IL03/00814. The guidewire of the present invention has such a small diameter that it may pass through any vasculature, even if occluded. The guidewire may be expanded to help break the occlusion. Thus, the guidewire can be used advantageously instead of a balloon (which cannot be made with such a small diameter) in PTCA procedures to open vasculature, and afterwards, a balloon may be placed in the vasculature which has been opened by the guidewire.

There is thus provided in accordance with an embodiment of the present invention a guidewire for a stent assembly, the guidewire including an outwardly expandable and inwardly contractible deformable portion.

In accordance with an embodiment of the present invention the deformable portion is formed on an outer slender tube, and a pull wire is disposed through the outer slender tube and connected to the deformable portion, wherein shifting the pull wire with respect to the outer slender tube moves the deformable portion between a collapsed orientation and an expanded orientation. For example, proximally shifting the pull wire with respect to the outer slender tube moves the deformable portion to the expanded orientation, and distally shifting the pull wire with respect to the outer slender tube moves the deformable portion to the collapsed orientation. The pull wire may be connected to a distal end of the deformable portion.

The deformable portion may include a plurality of support surfaces separated by cutouts. The support surfaces may be (but are not necessarily) contiguous and flush with the rest of the outer slender tube.

### BRIEF DESCRIPTION OF DRAWINGS

The present invention will be further understood and appreciated from the following detailed description taken in conjunction with the drawing in which:

FIG. 1 is a simplified pictorial illustration of a stent assembly, constructed and operative in accordance with an embodiment of the invention;

FIG. 2A is a simplified illustration of a guidewire used in the stent assembly of FIG. 1, including an outer slender tube with a pull wire disposed therein, constructed and operative in accordance with an embodiment of the invention;

FIG. 2B is a simplified illustration of the pull wire shifted proximally with respect to the outer slender tube, thereby bringing a deformable portion of the guidewire into an expanded orientation;

FIG. 2C is a simplified illustration of the guidewire, constructed and operative in accordance with another embodiment of the invention;

FIG. 3 is a simplified illustration of the stent assembly of FIG. 1 introduced into a body lumen that has a bifurcation, in accordance with an embodiment of the invention;

FIG. 4 is a simplified illustration of the stent assembly of FIG. 1 positioned in the body lumen such that the guidewire protrudes from a side aperture formed in the stent into a branch of the bifurcation, in accordance with an embodiment of the invention;

FIG. 5 is a simplified illustration of removing the distally removable portion and the proximally removable portion of the sheath of the stent assembly of FIG. 2, in accordance with an embodiment of the invention;

FIG. 6 is a simplified illustration of the stent assembly of FIG. 2, wherein the stent has expanded and the side aperture forms a flange at the bifurcation, in accordance with an embodiment of the invention;

FIG. 7 is a simplified illustration of the stent assembly of FIG. 2, wherein a branch stent has been introduced through the side aperture to the bifurcation, in accordance with an embodiment of the invention; and

FIG. 8 is a simplified illustration of the stent assembly of FIG. 2, wherein the branch stent is affixed to the stent of the stent assembly and is expanded in place in the bifurcation, in accordance with an embodiment of the invention.

### DETAILED DESCRIPTION OF EMBODIMENTS

Reference is now made to FIG. 1, which illustrates a stent assembly 25, constructed and operative in accordance with an embodiment of the invention. Stent assembly 25 may comprise a stent sheath 30 in which a stent 10 is initially disposed in a contracted orientation prior to deployment. Stent assembly 25 may be passed over guidewires to enter a body, as is described hereinbelow.

Stent 10 may be a wire mesh or braided stent, or any other kind of stent, but the invention is not limited to this construction. Stent 10 may be balloon-expandable, constructed from a suitable material, such as but not limited to, stainless steel 316L, or self-expanding, constructed from a suitable material, such as but not limited to, a shape memory alloy (such as a nickel-titanium alloy, e.g., NITINOL). Stent 10 may be formed with a side aperture 22 for placing therethrough a branch stent, as is described hereinbelow.

Sheath 30 may include two individually removable portions 32 and 34, which may be separable from one another. In the illustrated embodiment, sheath 30 comprises a distally removable portion 32 and a proximally removable portion 34. Distally removable portion 32 and proximally removable portion 34 may be completely separate from one another. Optionally, they may be initially joined by a rupturable element 36, such as but not limited to, a thin strip, wherein the rupturable element 36 may be severed, cut, ruptured, broken or otherwise removed so that the two portions 32 and 34 may be individually removed, as described hereinbelow. Distally removable portion 32 may comprise a distal cap 38, configured (e.g., preferably smooth and rounded) to facilitate movement of the stent assembly 25 in the vasculature.

Guidewires are preferably provided for manipulating portions of stent assembly 25. For example, a guidewire 40 may communicate with distally removable portion 32 and pass therethrough via an opening 23; a guidewire 42 may be attached to proximally removable portion 34; a guidewire 44 may be attached to stent 10, such as at a distal end thereof; and a guidewire 46 may be provided for passing through side aperture 22. The guidewires may be grasped and manipulated at the proximal end of a stent deployment catheter (not shown) as is well known in the art. In the illustrated embodiment, stent assembly 25 may be passed over guidewires 40 and 46 to reach a location in the body, as will be explained below.

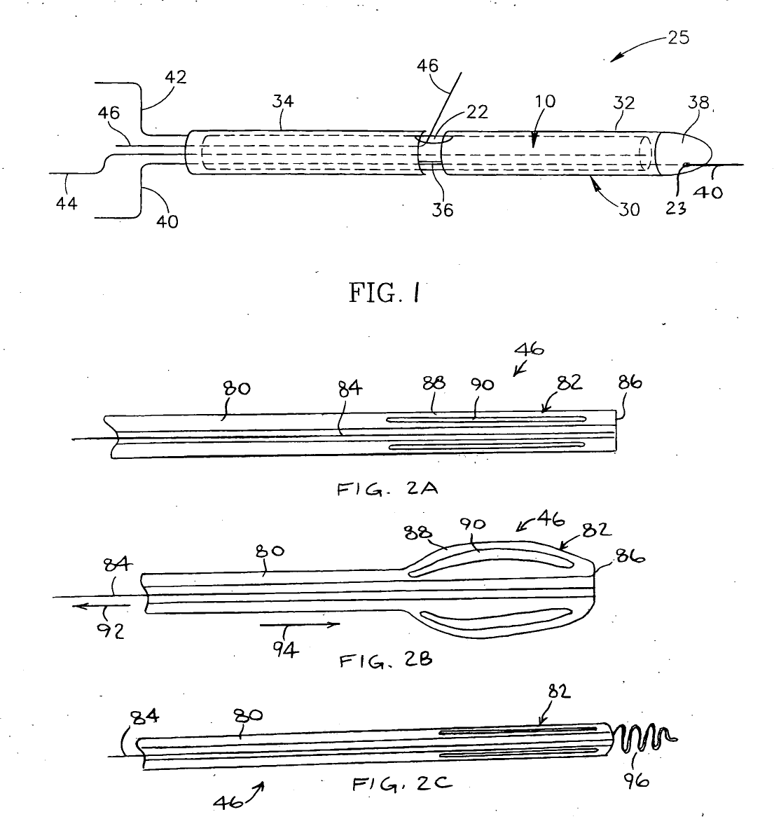

Reference is now made to FIG. 2A, which illustrates guidewire 46 more in detail. Guidewire 46 may include an outer slender tube 80 with a deformable portion 82. A slender pull wire 84 may be disposed through the length of outer slender tube 80 and connected to deformable portion 82, such as at a distal end 86 thereof. Outer slender tube 80 and its deformable portion 82 may be made of a durable material, such as but not limited to, a plastic, a shape memory alloy (such as a nickel-titanium alloy, e.g., NITINOL), or stainless steel which may be coated with a material such as polytetrafluoroethylene (PTFE). Guidewire 46 may have an outer diameter of about 0.38 mm, but the invention is not limited to this dimension.

Deformable portion 82 is shown in FIG. 2A in a collapsed orientation for placement in a body lumen, e.g., an artery. The deformable portion 82 may include a plurality of support surfaces 88 separated by cutouts 90. The support surfaces 88 may be of the same width as cutouts 90, or may be narrower or wider, depending on the application. The support surfaces 88 may be contiguous and flush with the rest of the outer slender tube 80. It is noted that although in the illustrated, non-limiting embodiment of FIG. 2A, outer slender tube 80 is generally cylindrical in shape, any other shape is also in the scope of the invention. The support surfaces 88 may be smooth or non-smooth, and may be coated with substances to obtain a desired effect. The support surfaces 88 may or may not be equally spaced.

The slender pull wire 84 may serve as an actuator to effect movement of the deformable portion 82 between a collapsed (or contracted, the terms being used interchangeably throughout) orientation and an expanded orientation. Referring now to FIG. 2B, pull wire 84 may be shifted proximally with respect to outer slender tube 80. For example, outer slender tube 80 may be held stationary and pull wire 84 may be pulled proximally in the direction of arrow 92 with respect to outer slender tube 80. Alternatively, pull wire 84 may be held stationary and outer slender tube 80 may be pushed distally in the direction of arrow 94 with respect to pull wire 84. Any of these actions brings deformable portion 82 into the expanded orientation shown in FIG. 2B. The reverse movement, that is, shifting pull wire 84 distally with respect to outer slender tube 80, may be used to return deformable portion 82 to the collapsed orientation of FIG. 2A.

It is emphasized that the invention is not limited to a pull wire as the actuator to effect movement of the deformable portion 82 between the collapsed and expanded orientations. Rather other actuators may be used to effect movement of the deformable portion 82 as well, such as but not limited to, mechanical (manual or motorized), pneumatic (e.g., balloon), hydraulic or any other expansion/contraction method.

Reference is now made to FIG. 2C, which illustrates another version of guidewire 46, constructed and operative in accordance with another embodiment of the invention. In this embodiment, guidewire 46 may include an elastic endpiece 96, such as but not limited to, a coil spring with a smooth (e.g., rounded) end. The elastic endpiece 96 may help negotiate tight turns in vasculature, for example.

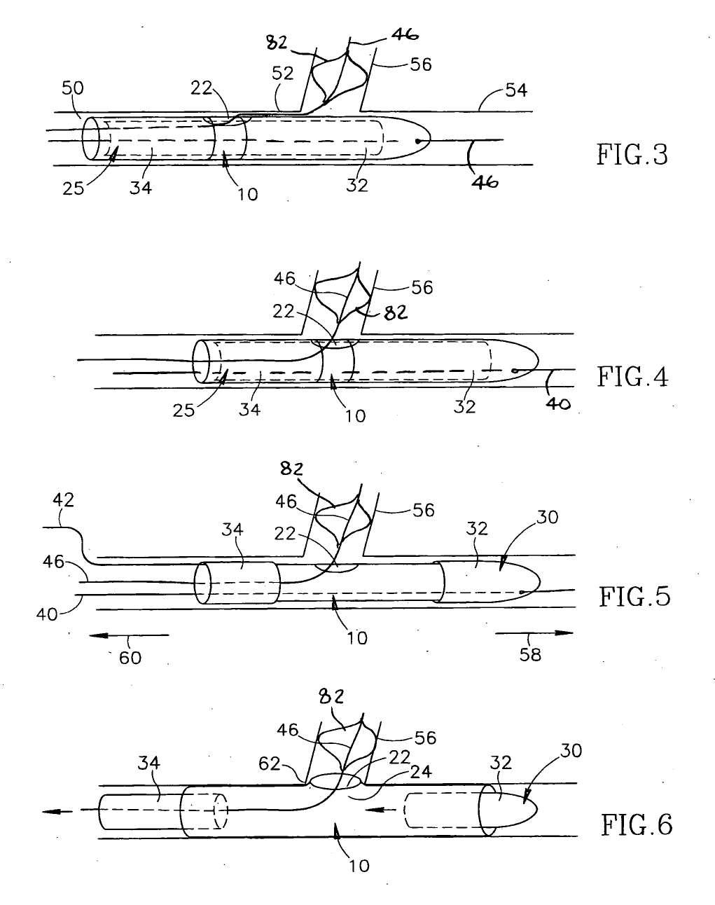

Reference is now made to FIG. 3, which illustrates introducing stent assembly 25 into a body lumen 50, in accordance with an embodiment of the invention. First, guidewires 40 and 46 may pass through vasculature until reaching the site of body lumen 50. Body lumen 50 may have a bifurcation comprising trunk 52 and branches 54 and 56. Guidewire 46 may be easily manipulated through side aperture 22 to enter branch 56. It is noted that guidewires 40 and 46 have such small diameters that they may pass through even occluded vasculature. The guidewire 46 may be expanded, for example, inside branch 56 to help break any occlusion in this branch. A stent deployment catheter (not shown) may be used to deliver stent assembly 25 into body lumen 50. Stent assembly 25 may glide over guidewires 40 and 46, such as in monorail fashion.

Reference is now made to FIG. 4, which illustrates stent assembly 25 positioned in body lumen 50 such that side aperture 22 is aligned with branch 56 of the bifurcation. Guide wire 46 protrudes from side aperture 22 and has been expanded so that it is anchored in branch 56. As described above, guidewire 46 may be expanded by shifting pull wire 84 proximally with respect to outer slender tube 80 in order to expand deformable portion 82 and anchor it against the walls of branch 56.

Reference is now made to FIG. 5, which illustrates one method of deploying stent 10 in the bifurcation. The distally removable portion 32 of sheath 30 may be removed by distally slipping (sliding) it off stent 10 by distally pushing with guidewire 40 (as indicated by an arrow 58). The proximally removable portion 34 of sheath 30 may be removed by proximally slipping (sliding) it off stent 10 by proximally pulling with guidewire 42 (as indicated by an arrow 60). Distally removable portion 32 and proximally removable portion 34 of sheath 30 may be removed simultaneously or one after the other or individually. (By individually it is meant that either one of the distally removable portion 32 and the proximally removable portion 34 is removed off stent 10 and the other removable portion is left on stent 10.)

After their removal, as seen in FIG. 6, sheath 10 expands and is affixed to the bifurcation, wherein aperture 22 is aligned with branch 56. Upon expansion of stent 10, a flange-forming structure 24 at aperture 22 may expand to form a flange 62 which may hug and overlap the juncture of branch 56 with the bifurcation. The distally removable portion 32 of sheath 30 may be removed from the vasculature by pulling it proximally through stent 10, since the expanded stent 10 now has a larger diameter than the sheath 30. The proximally removable portion 34 of sheath 30 may also be removed from the vasculature.

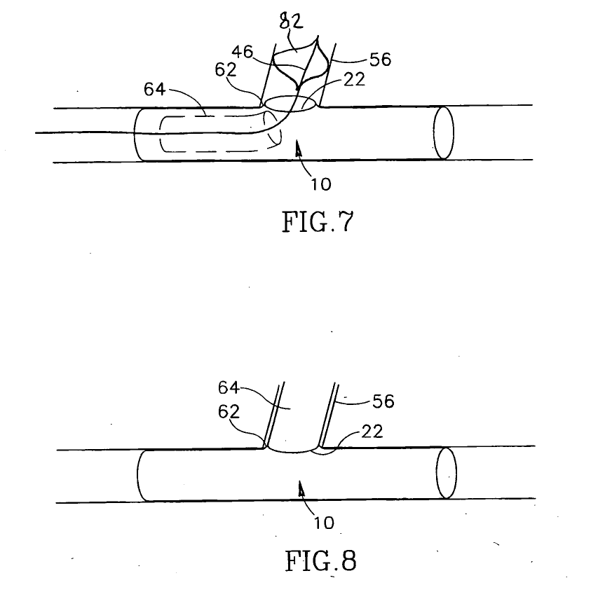

Reference is now made to FIG. 7, which illustrates introducing a branch stent 64 through side aperture 22 to the bifurcation. The branch stent 64 may also be a self-expanding wire mesh stent constructed from a shape memory alloy, but the invention is not limited to this construction. The branch stent 64 may be introduced with a conventional sheath and catheter (not shown) as well known in the art.

Reference is now made to FIG. 8, which illustrates branch stent 64 expanded in place in branch 56. Branch stent 64 may be affixed to flange 62 of stent 10. For example, branch stent 64 may snap-fit or press-fit together with flange 62, or by any other joining means.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

1. A guidewire for a stent assembly, the guidewire comprising an outwardly expandable and inwardly contractible deformable portion.

2. The guidewire according to claim 1, wherein said deformable portion is formed on an outer slender tube, and a pull wire is disposed through said outer slender tube and connected to said deformable portion, wherein shifting said pull wire with respect to said outer slender tube moves said deformable portion between a collapsed orientation and an expanded orientation.

3. The guidewire according to claim 2, wherein proximally shifting said pull wire with respect to said outer slender tube moves said deformable portion to the expanded orientation, and distally shifting said pull wire with respect to said outer slender tube moves said deformable portion to the collapsed orientation.

4. The guidewire according to claim 2, wherein said pull wire is connected to a distal end of said deformable portion.

5. The guidewire according to claim 1, wherein said deformable portion comprises a plurality of support surfaces separated by cutouts.

6. The guidewire according to claim 2, wherein said support surfaces are contiguous and flush with the rest of the outer slender tube.

7. The guidewire according to claim 1, wherein said guidewire includes an elastic endpiece.

8. A method comprising:
providing a guidewire that includes an outwardly expandable and inwardly contractible deformable portion;
manipulating said guidewire through vasculature to an occlusion; and
expanding said guidewire so as to break said occlusion.

9. The method according to claim 8, further comprising expanding said guidewire to anchor it in vasculature.
